(19) United States
(12) Patent Application Publication
(10) Pub. No.: US 2023/0077519 A1
(43) Pub. Date: Mar. 16, 2023

(54) SYSTEM AND METHOD FOR PROVIDING CONTEXT-BASED LIGHT AND/OR AUDITORY STIMULUS EXPERIENCE

(71) Applicant: INTO Technologies Inc., San Francisco, CA (US)

(72) Inventors: Justin Cheung, Pymble (AU); William Meeker, San Francisco, CA (US)

(21) Appl. No.: 17/939,764

(22) Filed: Sep. 7, 2022

Related U.S. Application Data
(60) Provisional application No. 63/241,833, filed on Sep. 8, 2021.

(51) Int. Cl. A61M 21/02; A61B 5/00; A61B 5/11

(52) U.S. Cl. CPC A61M 21/02; A61B 5/4815; A61B 5/1118; A61M 2021/0027

(57) ABSTRACT

A system and method for providing a context-based light and/or auditory stimulus experience to a user that includes administering dosages of light is disclosed. Environmental contextual data and/or personal (e.g., biometric and/or non-biometric) contextual data of a subject are obtained. Specific dosages of light are delivered to a subject based on the obtained environmental and/or personal contextual data. The dosages may be defined according to various parameters, including light wavelength, pulse frequency, intensity, area within the subject's field of vision, duration, pulse waveform shape, or a combination thereof. Auditory stimulus may also be provided, in synchronization with the administered dosages of light, and may be based on the obtained environmental and/or personal contextual data.

### CROSS-REFERENCE TO RELATED APPLICATIONS

This application claims priority to U.S. Provisional Patent Application No. 63/241,833, filed Sep. 8, 2021.

This application incorporates by reference U.S. Provisional Patent Application No. 62/877,602, filed Jul. 23, 2019, U.S. Provisional Patent Application No. 62/961,435, filed Jan. 15, 2020, U.S. Provisional Patent Application No. 63/049,203, filed Jul. 8, 2020, U.S. Non-Provisional patent application Ser. No. 16/937,124, filed Jul. 23, 2020, International Application No. PCT/US20/43324, filed Jul. 23, 2020, U.S. Provisional Patent Application No. 63/171,900, filed Apr. 7, 2021, and U.S. Provisional Patent Application No. 63/241,833, filed Sep. 8, 2021, in their entireties.

### TECHNICAL FIELD

The present disclosure relates to systems and methods for providing a context-based light and/or auditory stimulus experience to a user that includes administering dosages of light.

### BACKGROUND

A need exists to provide a light and/or auditory stimulus experience, including administering dosages of light, which is tailored based on environmental and/or biometric contexts.

### BRIEF SUMMARY

Disclosed are systems and methods of providing a context-based light and/or auditory stimulus experience to a user that includes administering dosages of light.

### BRIEF DESCRIPTION OF DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 illustrates a system, according to an embodiment.

FIG. 2 illustrates a sensor sub-system, according to an embodiment.

FIG. 3A illustrates an environmental sensor set, and FIG. 3B illustrates a biometric sensor set, according to an embodiment.

FIG. 4 illustrates an example computer system.

FIG. 5 illustrates an exemplary flow chart for selecting existing content using contextual input(s), according to an embodiment.

FIG. 6 illustrates an exemplary flow chart for selecting existing content using contextual input(s), according to an embodiment.

FIG. 7 illustrates an exemplary flow chart for modifying existing content using contextual input(s), according to an embodiment.

FIG. 8 illustrates an exemplary flow chart for modifying existing content using contextual input(s), according to an embodiment.

FIG. 9 illustrates an exemplary flow chart for generating content using contextual input(s), according to an embodiment.

FIG. 10 illustrates an exemplary flow chart for generating content using contextual input(s), according to an embodiment.

### DETAILED DESCRIPTION

The present disclosure relates to systems and methods of providing a context-based light and/or auditory stimulus experience to a user, based on contextual inputs such as environmental and/or biometric contexts. Exemplary devices/systems are described herein, and additional devices/systems that may be used in combination with the methods described herein are described in U.S. Provisional Patent Application No. 62/877,602, filed Jul. 23, 2019, U.S. Provisional Patent Application No. 62/961,435, filed Jan. 15, 2020, U.S. Provisional Application No. 63/049,203, filed Jul. 8, 2020, U.S. Non-Provisional patent application Ser. No. 16/937,124, filed Jul. 23, 2020, International Application No. PCT/US20/43324, filed Jul. 23, 2020, and U.S. Provisional Patent Application No. 63/171,900, filed Apr. 7, 2021, each of which are hereby incorporated by reference in their entireties, and are also described herein. While specific exemplary devices/systems are described or incorporated by reference herein, it should be understood that this is done for illustration purposes only. Other components and configurations may be used without departing from the spirit and scope of the invention.

The systems and methods of the present invention may be employed to alter a brain state of a user and improve the user's mental or physical functions. The systems and methods of the present invention may also be employed to treat various medical indications. The systems and methods of the present invention may include administering one or more dosage(s) of light to a user. The dosage(s) of light may be administered to the user's eyes, so as to stimulate the user's retinal ganglion cells within the user's eyes. The dosage(s) of light may be administered while the user's eyes are closed, by transmitting the light dosage(s) through the user's eyelids. The light dosage(s) may be defined according to various parameters including wavelength, area within the user's field of vision, intensity, pulse frequency, duration, pulse waveform shape, photon quantity, or any combination thereof.

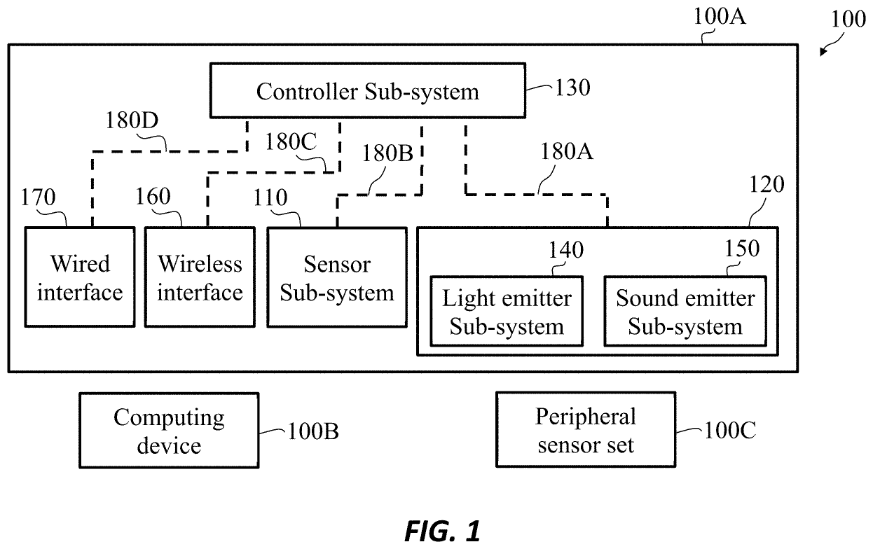

FIG. 1 illustrates a system 100 according to an example embodiment, which may be used in combination with the methods of providing a context-based light and/or auditory stimulus experience to a user as disclosed herein. In one embodiment, the system 100 includes a stimulus delivery device 100A, a computing device 100B, and a peripheral sensor set 100C.

In one embodiment, the stimulus delivery device 100A is configured to emit a light and/or auditory stimulus to a user. According to an embodiment, the stimulus delivery device 100A may include a sensor sub-system 110, an emitter sub-system 120, a controller sub-system 130, a wireless interface 160, and a wired interface 170. Additional aspects of these features are described in more detail below.

In one embodiment, the computing device 100B provides computing capabilities and an interface for a user of the system 100. The computing device 100B provides computing functions based on an operating system that executes various applications. In one embodiment, the computing device 100B communicates with the stimulus delivery device 100A, and optionally may also communicate with the external sensor set 100C. In one embodiment, the computing device 100B is a smartphone, such as, for example, a smartphone running the Apple iOS operating system or Google Android operating system. In one embodiment, the computing device 100B is a computer (e.g., desktop or laptop).

In one embodiment, the external sensor set 100C includes various sensors. The external sensor set 100C may include various environmental sensors including, but not limited to, an ambient natural light sensor, an ambient artificial light sensor, a UV light sensor, an ambient noise sensor, an ambient temperature sensor, a humidity sensor, a proximity sensor, and a GPS sensor.

The peripheral sensor set 100C may also, or alternatively, include various biometric sensors to collect biometric data of a user, such biometric sensors including, but not limited to, an electromyography (EMG) sensor, an electroencephalogram (EEG) sensor, a heart rate and/or heart rate variability sensor, an oxygen saturation sensor, a galvanic skin response sensor, a blood pressure sensor, a body temperature sensor, a blood glucose sensor, a step count sensor, and a respiratory rate sensor.

In one embodiment, one or more sensors within the peripheral sensor set 100C communicates with the stimulus delivery device 100A. For example, some or all sensors within the peripheral sensor set 100C may have a Bluetooth or WiFi transceiver or a USB interface for transmitting collected sensor data to the stimulus delivery device 100A.

In one embodiment, one or more sensors within the peripheral sensor set 100C communicates with the computing device 100B. For example, some or all sensors within the peripheral sensor set 100C may have a Bluetooth or WiFi transceiver or a USB interface for transmitting collected sensor data to the computing device 100B.

In one embodiment, one or more sensors within the peripheral sensor set 100C communicates with the Internet (e.g., over a WiFi connection). For example, some or all sensors within the peripheral sensor set 100C may have a WiFi transceiver or Ethernet port for transmitting collected sensor data to a server over the Internet.

In one embodiment, different sensors within the external sensor set 100C communicate with different devices. For example, one or more sensors within the external sensor set 100C may communicate with the stimulus delivery device 100A, one or more other sensors within the external sensor set 100C may communicate with the computing device 100B, and one or more still other sensors within the external sensor set 100C may communicate with the Internet (e.g., via WiFi).

### Emitter Sub-System

The emitter sub-system 120 may be in communication with the controller sub-system 130 via a data link 180A. As explained in more detail below, the system 100 may be configured to apply light and/or auditory stimulus to the user via the emitter sub-system 120, based on control by the controller sub-system 130 according to a light/sound stimuli control program (also referenced herein as “content” or an “experience”) defining the operation of the emitter sub-system 120.

The emitter sub-system 120 may include one or more stimulus emitters. For example, the emitter sub-system 120 may include a light emitter sub-system 140 to emit a light stimulus, and a sound emitter sub-system 150 to emit an auditory stimulus. These sub-systems are explained in detail below. In one embodiment, the light emitter sub-system 140 may be configured to apply one or more predetermined dosages of light to the user, to stimulate the user's retinal ganglion cells within the user's eyes. The predetermined dosages of light, for example, may be defined according to various parameters including:
(i) one or more predetermined wavelengths of light,
(ii) one or more predetermined areas/zones within the user's field of vision to direct the emitted light, including one or more individual areas/zones for each predetermined wavelength,
(iii) one or more predetermined intensities or amplitudes of light, including one or more individual intensities for each predetermined wavelength and/or area/zone,
(iv) one or more predetermined pulse frequencies or pulse rates (Hz), including one or more individual pulse frequencies for each predetermined wavelength, area/zone, and/or intensity,
(v) one or more durations to emit each wavelength, area/zone, intensity, and/or pulse frequency of light,
(vi) pulse waveform shape(s) (e.g., square wave, sine wave, sawtooth wave with a falling edge, sawtooth wave with a rising edge, etc.),
(vii) the quantity of photons being emitted to a user, which may depend on one or more of the above parameters, and/or
(viii) any combination of these parameters and/or other parameters.

In one embodiment, the sound emitter sub-system 150 may be configured to apply a predetermined auditory stimulus to the user. The predetermined auditory stimulus may be defined according to various parameters including:
(i) one or more predetermined beat frequencies (including any frequency and/or phase offset between the beat frequencies and the pulse frequencies emitted by the light emitter sub-system 140),
(ii) individual beat frequencies between different audio channels (e.g., left and right channels, such as to emit binaural beats),
(iii) audio frequencies of the one or more beat frequencies, including one or more individual audio frequencies for each predetermined beat frequency and/or audio channel,
(iv) one or more predetermined intensities or amplitudes of the beat frequencies, including one or more individual intensities for each predetermined beat frequency, audio channel, and/or audio frequency,
(v) one or more durations to emit each beat frequency, audio frequency, and/or intensity for each audio channel,
(vi) pulse waveform shape,
(vii) other audio emission (e.g., musical accompaniment to the beat(s)), and/or
(viii) any combination of these parameters and/or other parameters.

The controller sub-system 130 may control the emitter sub-system 120 including the light emitter sub-system 140 and/or the sound emitter sub-system 150, to control the attributes of light and/or sound stimuli to the user.

According to an embodiment, additional stimuli beyond light and auditory may be employed in the system.

### Light Emitter Sub-System

The light emitter sub-system 140 may include one or more lights to deliver light-based stimulus to the user. The one or more lights may be, for example, a micro-light emitting diode (micro-LED) or LED configured to controllably emit light according to the parameters described above, based on control by the controller sub-system 130.

In one embodiment, the lights of the light emitter sub-system 140 may be configured as single-wavelength or narrow-wavelength emitters (e.g., LEDs) distributed in a predetermined arrangement so as to direct light of specific wavelength(s) (or narrow wavelength bands) to a specific field/zone of vision of the user.

In one embodiment, the light emitters are distributed in a non-uniform manner along the field/zone of vision of a user. In one embodiment, light emitters (e.g., single-wavelength emitters) emitting ultraviolet and/or purple light wavelengths are distributed with greater concentration at regions corresponding to a peripheral field/zone of vision of the user, compared to regions corresponding to a central field/zone of vision. In one embodiment, light emitters (e.g., single-wavelength emitters) emitting ultraviolet and/or purple light wavelengths are only provided at regions corresponding to a peripheral field/zone of vision of the user, and are not provided at regions corresponding to a central field/zone of vision. In one embodiment, light emitters (e.g., single-wavelength emitters) emitting red and/or infrared light wavelengths are distributed with greater concentration at regions corresponding to a central field/zone of vision of the user, compared to regions corresponding to a peripheral field/zone of vision. In one embodiment, light emitters (e.g., single-wavelength emitters) emitting red and/or infrared light wavelengths are only provided at regions corresponding to a central field/zone of vision of the user, and are not provided at regions corresponding to a peripheral field/zone of vision. In one embodiment, the light emitters are arranged in a left-right symmetrical pattern.

In one embodiment, the light emitters (e.g., single-wavelength emitters) are grouped according to their emitted wavelengths. In one embodiment, the number of groups (i.e., the number of emitted single wavelengths or narrow wavelength bands) is greater than 3. In one embodiment, the number of groups is greater than 4. In one embodiment, the number of groups is in a range between 4 and 16. In one embodiment, the number of groups is in a range between 6 and 10. In one embodiment, the number of groups is 8.

In one embodiment, the light emitters within each group may all be identical to one another. In one embodiment, at least two light emitters within an individual group may differ from one another.

In one embodiment, the groups of single-wavelength emitters correspond to respective color channels controlled by the controller sub-system 130. In one embodiment, the light emitter sub-subsystem 140 may contain 192 LEDs, split into 8 color channels, where each color channel corresponds to a different peak wavelength and has 24 identical LEDs. In one embodiment, the light emitter sub-system 140 may include 24 LEDs in each color channel, which is split into 3 smaller “pixels” of 8 identical LEDs in series. In one embodiment, the 24 “pixels” (i.e., 8 channels of 3 pixels) for each color channel are driven by a pulse width modulation (PWM) constant-current sink LED driver with an internal oscillator. The PWM driver may provide PWM control to each pixel based on a grayscale value for an individual “frame” of an experience to be provided to the user. In one embodiment, the controller sub-system 130 may provide all 3 “pixels” within a given color with the same control information (e.g., without further splitting the pixels into smaller spatial zones). Of course, it will be appreciated that the device may include a different number of color channels, a different grouping of “pixels”, a different number of total light emitters per color channel, and/or other different characteristics than those exemplary characteristics described herein.

In one embodiment, the light emitters are selected to have a narrow spectral output and to collectively summarize the visible spectrum. In one embodiment where the number of color channels is 8, the corresponding channels of wavelengths or narrow wavelength bands may be:

| | | Viewing | Luminous | |
|---|---|---|---|---|
| | Wavelength | Angle | Intensity | |
| Nominal color | (nm) | (deg) | (mcd) | Number |
| Ultraviolet | 390-395 | 130 | — | 24 |
| Purple/UV 2 | 405-410 | 130 | — | 24 |
| Blue | 468 | 130 | 115 | 24 |
| Green | 515 | 140 | 430 | 24 |
| True Green | 530 | 120 | 350 | 24 |
| Red | 639 | 130 | 54 | 24 |
| Deep Red | 660 | 140 | 16 | 24 |
| Far Red/IR | 700 | 140 | 5 | 24 |

In one embodiment, where the number of color channels is 8, the corresponding channels of wavelengths or narrow wavelength bands may be:

| | | Viewing | Luminous | |
|---|---|---|---|---|
| | Wavelength | Angle | Intensity | |
| Nominal color | (nm) | (deg) | (mcd) | Number |
| Violet 1 | 405 | 130 | — | 24 |
| Violet 2 | 415 | 150 | — | 24 |
| Blue | 468 | 130 | 115 | 24 |
| Green | 515 | 140 | 430 | 24 |
| True Green | 530 | 120 | 350 | 24 |
| Red | 639 | 130 | 54 | 24 |
| Deep Red | 660 | 140 | 16 | 24 |
| Far Red/IR | 700 | 140 | 5 | 24 |

The arrangements of single-wavelength emitters may provide the capability to deliver light dosages of specific wavelengths to specific areas of a user's field of vision, while also providing a greater concentration of light emitters of particular wavelengths in certain regions (e.g., violet or ultraviolet wavelengths more concentrated at a peripheral region).

In one embodiment, at least a subset of the single wavelengths or narrow wavelength bands emitted by the light emitter sub-systems are beyond the visually perceptible range for humans. In one embodiment, the light emitter sub-system 120 and/or controller sub-system 130 are configured to emit the dosage(s) of light at least partially while the user's eyes are open. In one embodiment, the light emitter sub-system 120 and/or controller sub-system 130 are configured to emit the dosage(s) of light while the user's eyes are closed. In one embodiment, the light emitter sub-system 120 and/or controller sub-system 130 are configured to emit the dosage(s) of light only while the user's eyes are closed.

### Sound Emitter Sub-System

According to an embodiment, the sound emitter sub-system 150 may include one or more speakers to deliver auditory stimulus to the user. The sound emitter sub-system 150 may alternatively or additionally include one or more interfaces (e.g., 3.5 mm audio jack, RCA or digital audio jacks, or Bluetooth) allowing the connection of peripheral audio components (e.g., headphones) for emitting the auditory stimulus to the user. For instance, wired or wireless headphones may be used for delivering binaural-beat auditory stimulus to the user. The sound emitter sub-system 150 may be configured to controllably emit audio according to the parameters described above, based on control by the controller sub-system 130.

### Sensor Sub-System

The sensor sub-system 110 may be in communication with the controller sub-system 130 via a data link 180B. As explained in more detail below, the system 100 may be configured to receive data from one or more sensors.

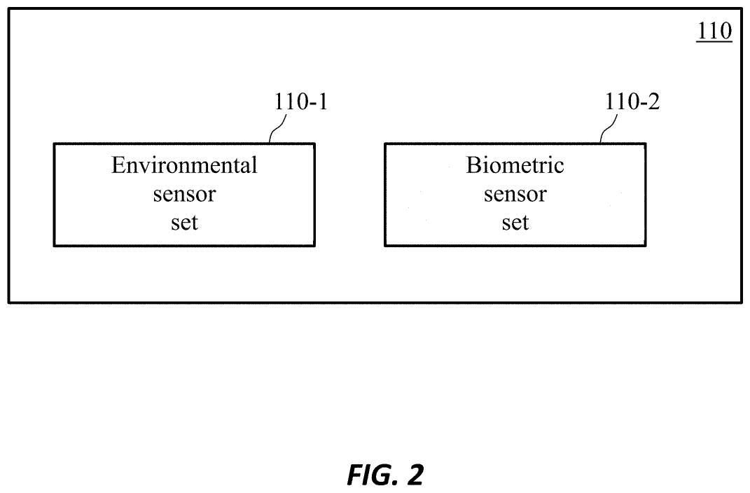

FIG. 2A illustrates the sensor sub-system 110 according to an example embodiment. The sensor sub-system 110 may include an environmental sensor set 110-1 that includes one or more environmental sensors, and a biometric sensor set 110-2 that includes one or more biometric sensors. Further details of these sensor sets will be described below. In one embodiment, one or more of the sensors in these sensor sets is physically integrated with the system. In one embodiment, one or more of the sensors in these sensor sets is an external sensor (e.g., third-party sensor) that communicates with the controller sub-system 130 via the wireless interface 160 and/or the wired interface 170.

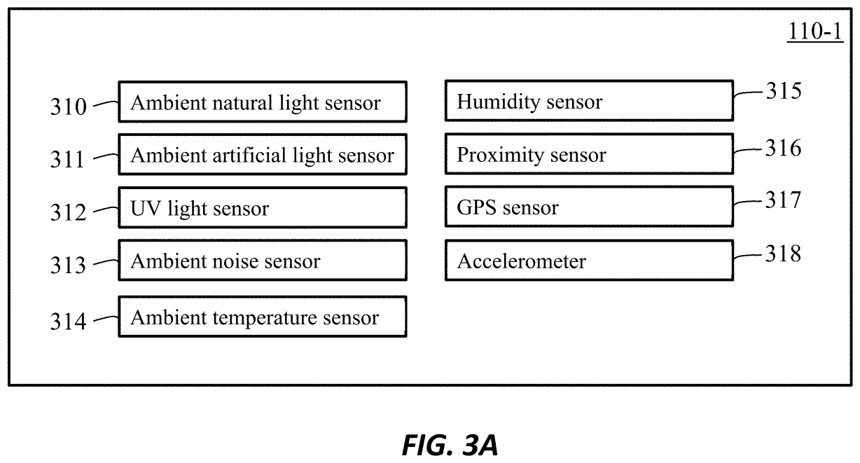

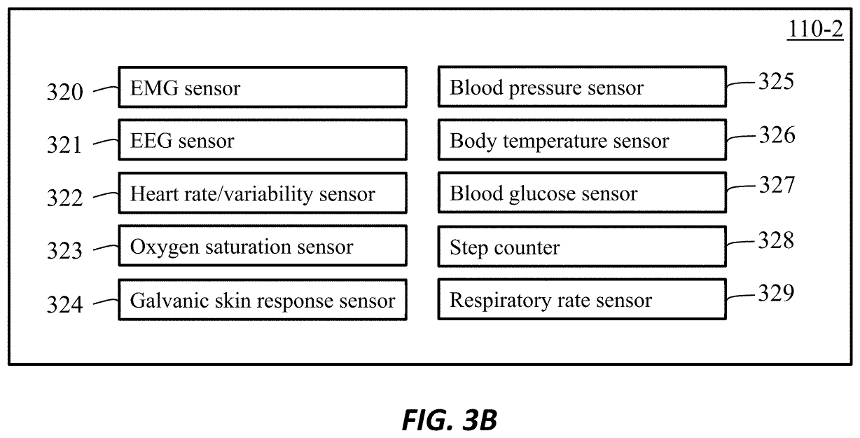

FIG. 3A illustrates the environmental sensor set 110-1 according to an embodiment. The environmental set 110-1 may include one or more of an ambient natural light sensor 310, an ambient artificial light sensor 311, a UV light sensor 312, an ambient noise sensor 313, an ambient temperature sensor 314, a humidity sensor 315, a proximity sensor 316, a GPS sensor 317, and an accelerometer 318.

The ambient natural light sensor 310 may detect a level of ambient natural light (e.g., sunlight) of an area corresponding to the system 100. The ambient natural light sensor 310 may include functionality to distinguish natural light from artificial light.

The ambient artificial light sensor 311 may detect a level of ambient artificial light (e.g., incandescent, fluorescent, and/or LED) of an area corresponding to the system 100. The ambient artificial light sensor 311 may include functionality to distinguish natural light from artificial light.

The UV light sensor 312 may detect a level of ultraviolet (UV) light of an area corresponding to the system 100. The UV light sensor 312 may include functionality (e.g., filter) to distinguish UV light from other light.

In one embodiment, one or more of the ambient natural light sensor 310, the ambient artificial light sensor 311, and the UV light sensor 312 may be implemented as one or more photometers, one or more image sensors, and/or one or more spectrometers. Of course, it will be appreciated that the ambient natural light sensor 310, the ambient artificial light sensor 311, and/or the UV light sensor 312 may be implemented using any other known component for measuring light levels. It will also be appreciated that the ambient natural light sensor 310, the ambient artificial light sensor 311, and/or the UV light sensor 312 may be integrated as a single sensor or may be provided as separate sensors.

The ambient noise sensor 313 may detect a level of ambient noise of an area corresponding to the system 100. In one embodiment, the ambient noise sensor 313 includes one or more microphones.

The ambient temperature sensor 314 may detect the ambient temperature of an area corresponding to the system 100. In one embodiment, the ambient temperature sensor 314 includes one or more thermistors.

The humidity sensor 315 may detect the absolute and/or relative humidity of an area corresponding to the system 100. In one embodiment, the humidity sensor 315 is a hygrometer. In one embodiment, a component within the system 100 (e.g., the controller sub-system 130 or the humidity sensor 315 itself) may determine a relative humidity level based on an absolute humidity measurement and an ambient temperature measurement (e.g., from the ambient temperature sensor 314).

The proximity sensor 316 may detect the presence and/or proximity of other individuals in an area corresponding to the system 100. It will be appreciated that the proximity sensor 316 may be implemented as a separate sensor or may be implemented in software/firmware based on measurement data from one or more of the other sensors. As one non-limiting example, the presence and/or proximity of other individuals in the area may be determined based on the volume and/or number of detected voices from measurements provided by the ambient noise sensor 313.

The GPS sensor 317 may detect GPS signals and determine a geographical location and standardized time and/or local time based on the determined geographical location.

The accelerometer 318 may detect acceleration and/or movement of the system 100.

FIG. 3B illustrates the biometric sensor set 110-2 according to an embodiment. The biometric sensor set 110-2 may include an electromyography (EMG) sensor 320, an electroencephalogram (EEG) sensor 321, a heart rate and/or heart rate variability sensor 322, an oxygen saturation sensor 323, a galvanic skin response sensor 324, a blood pressure sensor 325, a body temperature sensor 326, a blood glucose sensor 327, a step counter 328, and a respiratory rate sensor 329.

The electromyography (EMG) sensor 320 may detect electrical activity produced by a muscle response of a user of the system 100.

The electroencephalogram (EEG) sensor 321 may detect electrical activity produced by brain waves of a user of the system 100.

The heart rate/variability sensor 322 may detect the heart rate and/or the heart rate variability of a user of the system 100.

The oxygen saturation sensor 323 may detect the blood oxygen level of a user of the system 100.

The galvanic skin response sensor 324 may detect the sweat gland activity of a user of the system 100.

The blood pressure sensor 325 may detect the blood pressure level of a user of the system 100.

The body temperature sensor 326 may detect the body temperature of a user of the system 100.

The blood glucose sensor 327 may detect the blood glucose level of a user of the system 100.

The step counter 328 may detect a user's movement (e.g., number of steps).

The respiratory rate sensor 329 may detect a user's respiratory rate.

It will be appreciated the described sensors in the environmental sensor set 110-1 and the biometric sensor set 110-2 are merely exemplary, that not all of the sensors are required, that the system may be formed using a subset of the sensors described above, and that the system may include additional sensors or sensor sets beyond those described herein.

### Wireless Interface

The wireless interface 160 may be in communication with the controller sub-system 130 via a data link 180C. The wireless interface 160 may provide communication between the system 100 and external devices (e.g., peripherals) including, but not limited to, sensors, smartphones, computers, fitness trackers, and various peripherals, and may also provide Internet connectivity for the system 100. In one embodiment, the wireless interface 160 may include one or more of a WiFi transceiver, Bluetooth transceiver, ANT+transceiver, and/or NFC transceiver.

In one embodiment, the WiFi transceiver of the wireless interface 160 may wirelessly communicate over the Internet via a wireless access point, to transmit and/or receive data from an Internet server. In one embodiment, the WiFi transceiver of the wireless interface 160 may wirelessly transmit data to, and/or receive data from, a device on the same local network as the system 100 (e.g., a sensor device that transmits sensor data over WiFi to the system 100) or via an ad-hoc WiFi connection.

In one embodiment, the Bluetooth transceiver of the wireless interface 160 may wirelessly transmit and/or receive data with a smartphone, such as via a dedicated application (also known as an “app”) loaded on the smartphone. In one embodiment, the Bluetooth transceiver of the wireless interface 160 may wirelessly transmit and/or receive data (e.g., sensor data) with a peripheral sensor device.

In one embodiment, the ANT+transceiver of the wireless interface 160 may wirelessly transmit and/or receive data with a peripheral sensor device (e.g., a sensor within the biometric sensor set 110-2, such as the heart rate/variability sensor 322).

### Wired Interface

The wired interface 170 may be in communication with the controller sub-system 130 via a data link 180D. The wired interface 170 may provide communication between the system 100 and external devices including, but not limited to, sensors, smartphones, computers, and various peripherals. The wired interface 170 may be a USB interface that includes a USB port (e.g., micro-USB or USB-C). In one embodiment, the wired interface 170 also provides power to the system 100. In one embodiment, the wired interface 170 provides power for charging a rechargeable power source (e.g., battery) in the system 100.

### Controller Sub-System

According to an embodiment, the controller sub-system 130 may utilize a general-purpose computing device 400, as explained in more detail below. In one embodiment, the controller sub-system 130 stores pre-programmed experiences of stimulus to present to a user, such as synchronized control sequences for the light emitter sub-system 140 and/or sound emitter sub-system 150, and controls these sub-systems accordingly to present the experience (e.g., including the defined dosing of light) to the user. In one embodiment, the controller sub-system 130 is configured to receive and store defined experiences (and/or modify existing stored experiences) based on information from an external source (e.g., over a network, from a USB storage device, based on user input and/or control parameters, etc.).

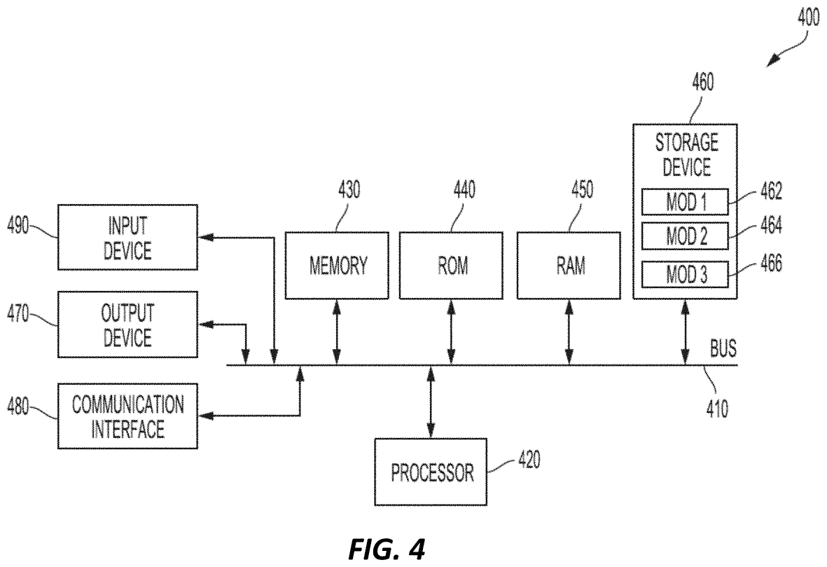

With reference to FIG. 4, an exemplary arrangement of the controller sub-system 130 described above includes a general-purpose computing device 400, including a processing unit (CPU or processor) 420 and a system bus 410 that couples various system components including the system memory 430 such as read-only memory (ROM) 440 and random access memory (RAM) 450 to the processor 420. The system 400 can include a cache of high-speed memory connected directly with, in close proximity to, or integrated as part of the processor 420. The system 400 copies data from the memory 430 and/or the storage device 460 to the cache for quick access by the processor 420. In this way, the cache provides a performance boost that avoids processor 420 delays while waiting for data. These and other modules can control or be configured to control the processor 420 to perform various actions. Other system memory 430 may be available for use as well. The memory 430 can include multiple different types of memory with different performance characteristics. It can be appreciated that the disclosure may operate on a computing device 400 with more than one processor 420 or on a group or cluster of computing devices networked together to provide greater processing capability. The processor 420 can include any general purpose processor and a hardware module or software module, such as module 1 462, module 2 464, and module 3 466 stored in storage device 460, configured to control the processor 420 as well as a special-purpose processor where software instructions are incorporated into the actual processor design. The processor 420 may essentially be a completely self-contained computing system, containing multiple cores or processors, a bus, memory controller, cache, etc. A multi-core processor may be symmetric or asymmetric.

The system bus 410 may be any of several types of bus structures including a memory bus or memory controller, a peripheral bus, and a local bus using any of a variety of bus architectures. A basic input/output (BIOS) stored in ROM 440 or the like, may provide the basic routine that helps to transfer information between elements within the computing device 400, such as during start-up. The computing device 400 further includes storage devices 460 such as a hard disk drive, a magnetic disk drive, an optical disk drive, tape drive or the like. The storage device 460 can include software modules 462, 464, 466 for controlling the processor 420. Other hardware or software modules are contemplated. The storage device 460 is connected to the system bus 410 by a drive interface. The drives and the associated computer-readable storage media provide nonvolatile storage of computer-readable instructions, data structures, program modules and other data for the computing device 400. In one aspect, a hardware module that performs a particular function includes the software component stored in a tangible computer-readable storage medium in connection with the necessary hardware components, such as the processor 420, bus 410, display 470, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the device 400 is a small, handheld computing device, a desktop computer, or a computer server.

Although the exemplary embodiment described herein employs the hard disk 460, other types of computer-readable media which can store data that are accessible by a computer, such as magnetic cassettes, flash memory cards, digital versatile disks, cartridges, random access memories (RAMs) 450, and read-only memory (ROM) 440, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 400, an input device 490 represents any number of input mechanisms, such as a microphone for speech, a touch-sensitive screen for gesture or graphical input, keyboard, mouse, motion input, speech and so forth. An output device 470 can also be one or more of a number of output mechanisms known to those of skill in the art. In some instances, multimodal systems enable a user to provide multiple types of input to communicate with the computing device 400. The communications interface 480 generally governs and manages the user input and system output. There is no restriction on operating on any particular hardware arrangement and therefore the basic features here may easily be substituted for improved hardware or firmware arrangements as they are developed.

### Methods of Selecting Existing Content using Contextual Input(s)

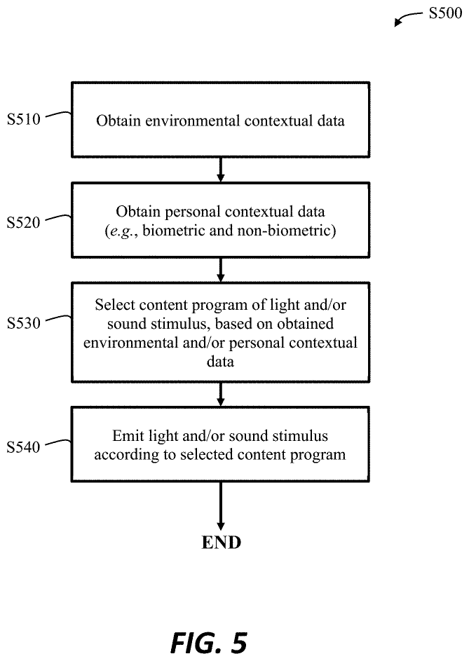

Various methods of selecting content using contextual input(s) according to the invention will now be described. FIG. 5 illustrates an embodiment of a process S500 for selecting existing content using contextual input(s), in accordance with the invention.

In step S510, the controller sub-system 130 of the stimulus delivery device 100A obtains environmental contextual data. Environmental contextual data, in the context of the invention described herein, may encompass data pertaining to the environment where the stimulus delivery device 100A is located. For example, environmental contextual data may include (but is not limited to) physical and/or situational attributes where the stimulus delivery device 100A is situated. For example, environmental contextual data may include (but is not limited to) data obtained from one or more sensors, obtained or inferred from geolocation, and/or input by a user. Such data is contextual as it may provide context in determining the content to emit to the user. In one embodiment, the obtained environmental contextual data includes one or more of time of day, weather, current geographical location, ambient natural light level, ambient artificial light level, ultraviolet light level, ambient noise level, ambient temperature, local outdoor temperature at the current geographical location, absolute and/or relative humidity, and proximity to other individuals.

In one embodiment, the controller sub-system 130 obtains some or all of the environmental contextual data from outputs from sensors within the environmental sensor set 110-1. For instance, the controller sub-system 130 may:
obtain the time of day and the current geographical location based on output data from the GPS sensor 317,
obtain the ambient natural light level based on output data from the ambient natural light sensor 310,
obtain the ambient artificial light level based on output data from the ambient artificial light sensor 311,
obtain the ultraviolet light level based on output data from the UV light sensor 312,
obtain the ambient noise level based on output data from the ambient noise sensor 313,
obtain the ambient temperature based on output data from the ambient temperature sensor 314,
obtain the absolute and/or relative humidity based on output data from the humidity sensor 315,
obtain weather information based on output data from the ambient temperature sensor 314 and/or the humidity sensor 315, and
obtain proximity to other individuals based on output data from the proximity sensor 316.

In one embodiment, the controller sub-system 130 of the stimulus delivery device 100A obtains some or all of the environmental contextual data from the computing device 100B. For instance, the controller sub-system 130 may be in communication with the computing device 100B via the wireless interface 160 and/or the wired interface 170, and may receive environmental contextual data obtained using an application executed on the computing device 100B. For instance, the application executed on the computing device 100B may:
obtain the time of day and the current geographical location by accessing system data (e.g., clock and/or location data) available on the computing device 100B,
obtain the ambient natural light level, ambient artificial light level, and/or ultraviolet light level by accessing output data from a light sensor and/or camera on the computing device 100B,
obtain the ambient noise level and/or proximity to other individuals by accessing audio data from a microphone on the computing device 100B, and
obtain the local outdoor temperature, absolute/relative humidity, and/or weather information at the current geographical location by (i) accessing location data available on the computing device 100B (e.g., via a built-in GPS sensor) and (ii) accessing an Internet portal that provides location-based temperature and/or weather information.

In addition (or as an alternative), the application executed on the computing device 100B may control communication between the computing device 100B and various sensors within the peripheral sensor set 100C, so as to obtain some or all of the environmental contextual data from the peripheral sensor set 100C. For instance, various sensors within the peripheral sensor set 100C may be in wireless (e.g., WiFi or Bluetooth) or wired (e.g., USB) communication with the computing device 100B and may transmit sensor data to the computing device 100B.

The application executed on the computing device 100B may in turn transmit such obtained information to the stimulus delivery device 100A via the wireless interface 160 (e.g., Bluetooth or WiFi connection) or the wired interface 170 (e.g., USB connection).

In one embodiment, the controller sub-system 130 obtains some or all of the environmental contextual data from the Internet, such as over a WiFi connection using the wireless interface 160. For instance, the controller sub-system 130 may:
obtain the time of day and the current geographical location by accessing such information on the Internet (e.g., an Internet portal providing time information and general geographical location based on IP address), and
obtain the local outdoor temperature, absolute/relative humidity, and/or weather information at the current geographical location by accessing an Internet portal that provides location-based temperature and/or weather information.

In addition (or as an alternative), the application executed on the computing device 100B may collect environmental contextual data, and provide such data to a server (e.g., via the Internet). The controller sub-system 130 may then download such collected contextual data from the Internet.

In one embodiment, the controller sub-system 130 obtains some or all of the environmental contextual data from the peripheral sensor set 100C. For instance, various sensors within the peripheral sensor set 100C may be in wireless or wired communication with the stimulus delivery device 100A via the wireless interface 160 or the wired interface 170, and may transmit sensor data to the stimulus delivery device 100A.

In one embodiment, the controller sub-system 130 obtains some or all of the environmental contextual data from third-party app data input, which may be acquired from the Internet and/or from the computing device 100B.

It will be appreciated that the environmental contextual data described herein are merely examples and that collected environmental contextual data is not limited to those examples described herein. A variety of other environmental contextual data may be contemplated for use with the invention herein.

In step S520, the controller sub-system 130 of the stimulus delivery device 100A obtains personal contextual data, such as biometric personal contextual data and/or non-biometric personal contextual data. Personal contextual data, in the context of the invention described herein, may encompass data pertaining to the individual to which the stimulus delivery device 100A emits the stimulus. For example, personal contextual data may include (but is not limited to) physical, biometric, health, activity, and/or situational attributes of the individual to which the stimulus delivery device 100A emits the stimulus. For example, personal contextual data may include (but is not limited to) data obtained from one or more sensors, inferred from history, and/or input by a user. Such data is contextual as it may provide context in determining the content to emit to the user. In one embodiment, the obtained biometric personal contextual data includes one or more of EMG measurements, EEG measurements, heart rate, heart rate variability, oxygen saturation level, galvanic skin response, blood pressure, body temperature, glucose level, respiratory rate, hormone levels, and sleep data (e.g., REM length, restlessness, etc.). The obtained biometric personal contextual data may also include fitness data (e.g., daily step count). The obtained non-biometric personal contextual data may include a user's personal calendar events.

In one embodiment, the controller sub-system 130 obtains some or all of the biometric personal contextual data from outputs from sensors within the biometric sensor set 110-2. For instance, the controller sub-system 130 may:
obtain a user's EMG measurement from the EMG sensor 320,
obtain a user's EEG measurement from the EEG sensor 321,
obtain a user's heart rate and/or heart rate variability from the heart rate/variability sensor 322,
obtain a user's oxygen saturation from the oxygen saturation sensor 323,
obtain a user's galvanic skin response from the galvanic skin response sensor 324,
obtain a user's blood pressure level from the blood pressure sensor 325,
obtain a user's body temperature from the body temperature sensor 326,
obtain a user's blood glucose level from the blood glucose sensor 327,
obtain a user's step count from the step count sensor 328, and
obtain a user's respiratory rate from the respiratory rate sensor 329.

In one embodiment, the controller sub-system 130 of the stimulus delivery device 100A obtains some or all of the personal contextual data from the computing device 100B. For instance, the application executed on the computing device 100B may:
obtain a user's heart rate, heart rate variability, oxygen saturation, and blood glucose level based on outputs from built-in sensors on the computing device 100B, and/or
obtain a user's calendar events from a calendar application on the computing device 100B.

In addition (or as an alternative), the application executed on the computing device 100B may control communication between the computing device 100B and various sensors within the peripheral sensor set 100C, so as to obtain some or all of the biometric personal contextual data from the peripheral sensor set 100C. For instance, various sensors within the peripheral sensor set 100C may be in wireless (e.g., WiFi or Bluetooth) or wired (e.g., USB) communication with the computing device 100B and may transmit sensor data to the computing device 100B. Various examples of such sensors that may be encompassed within the peripheral sensor set 100C include, but are not limited to, smartwatches, fitness bands/trackers, sleep trackers, and heart rate monitors that may communicate with the computing device 100B via WiFi, Bluetooth and/or ANT+protocols.

Furthermore (or as another alternative), the application executed on the computing device 100B may obtain some or all of the personal contextual data from the Internet. For instance, where a user utilizes a sensor within the peripheral sensor set 100C (e.g., fitness tracker) where collected biometric data is provided to an Internet server and is accessible via an Internet portal (e.g., by Apple, Fitbit, Garmin, etc.), the computing device 100B may obtain some or all of the biometric personal contextual data by accessing the Internet portal. And, where a user has a personal calendar accessible via an Internet portal, the computing device 100B may obtain some or all of the calendar data by accessing the Internet portal.

The application executed on the computing device 100B may in turn transmit such obtained information to the controller sub-system 130 via the wireless interface 160 (e.g., Bluetooth or WiFi connection) or the wired interface 170 (e.g., USB connection).

In one embodiment, the controller sub-system 130 obtains some or all of the personal contextual data from the Internet, such as over a WiFi connection using the wireless interface 160. For instance, where a user utilizes a sensor within the peripheral sensor set 100C (e.g., fitness tracker) where collected biometric data is provided to an Internet server and is accessible via an Internet portal (e.g., by Apple, Fitbit, Garmin, etc.), the controller sub-system 130 may obtain some or all of the biometric personal contextual data by accessing the Internet portal. And, where a user has a personal calendar accessible via an Internet portal, the controller sub-system 130 may obtain some or all of the calendar data by accessing the Internet portal.

In one embodiment, the controller sub-system 130 obtains some or all of the biometric personal contextual data from the peripheral sensor set 100C. For instance, various sensors within the peripheral sensor set 100C may be in wireless or wired communication with the stimulus delivery device 100A via the wireless interface 160 or the wired interface 170, and may transmit sensor data to the stimulus delivery device 100A. Various examples of such sensors that may be encompassed within the peripheral sensor set 100C include, but are not limited to, smartwatches fitness bands/trackers, sleep trackers, and heart rate monitors that may communicate with the stimulus delivery device 100A via WiFi, Bluetooth, and/or ANT+protocols.

In one embodiment, the controller sub-system 130 obtains some or all of the personal contextual data from third-party app data input, which may be acquired from the Internet and/or from the computing device 100B.

It will be appreciated that the obtained biometric personal contextual data may include current (e.g., real-time) biometric data, historical biometric data, or a combination of both.

In step S530, the controller sub-system 130 selects an ideal content program, based on the obtained environmental and personal contextual data. It will be appreciated that such selection may be implemented based on a variety of approaches. For example, based on the obtained local time, a content program having a particularly higher or lower dosage of blue light may be selected, given that blue light may interfere with natural circadian rhythms and cause insomnia. Based on the obtained ambient natural, ambient artificial, or ultraviolet light levels, a content program having a particularly higher or lower dosage intensity of light may be selected. Based on the obtained ambient noise level or proximity information, a content program having a particularly higher or lower audio intensity or range may be selected. Based on EMG, EEG, heart rate, blood pressure, or respiratory rate, a content program designed to provide a calming or soothing experience or, conversely, a content program designed to increase alertness and/or reduce drowsiness, may be selected.

In one embodiment, the controller sub-system 130 determines an ideal content program by transmitting collected contextual information to a server (e.g., via the Internet) and receiving an identification of a content program already stored on the controller sub-system 130 and/or receives the content program itself.

In step S540, the controller sub-system 130 controls the emitter sub-system 120 to emit light and/or sound according to the selected content program.

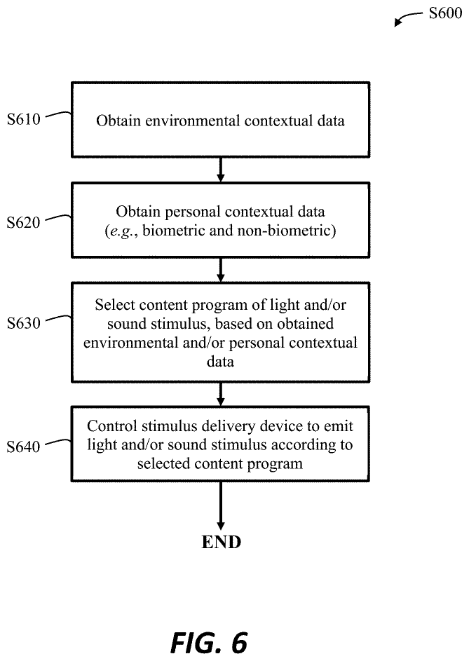

FIG. 6 illustrates an embodiment of another process S600 for selecting existing content using contextual input(s), in accordance with the invention. The process S600 differs from the process S500 primarily in that the computing device 100B, instead of the controller sub-system 130, performs many of the steps. For instance, a stimulus control application running on the computing device 100B performs many of the steps.

In step S610, the stimulus control application of the computing device 100B obtains environmental contextual data. In one embodiment, the obtained environmental contextual data includes one or more of time of day, weather, current geographical location, holidays, lunar/solar events, ambient natural light level, ambient artificial light level, ultraviolet light level, ambient noise level, ambient temperature, local outdoor temperature at the current geographical location, absolute and/or relative humidity, and proximity to other individuals.

In one embodiment, the stimulus control application of the computing device 100B obtains some or all of the environmental contextual data from outputs from sensors within the environmental sensor set 110-1 of the stimulus delivery device 100A. For instance, the controller sub-system 130 of the stimulus delivery device 100A may:
obtain the time of day and the current geographical location based on output data from the GPS sensor 317,
obtain the ambient natural light level based on output data from the ambient natural light sensor 310,
obtain the ambient artificial light level based on output data from the ambient artificial light sensor 311,
obtain the ultraviolet light level based on output data from the UV light sensor 312,
obtain the ambient noise level based on output data from the ambient noise sensor 313,
obtain the ambient temperature based on output data from the ambient temperature sensor 314,
obtain the absolute and/or relative humidity based on output data from the humidity sensor 315,
obtain weather information based on output data from the ambient temperature sensor 314 and/or the humidity sensor 315, and
obtain proximity to other individuals based on output data from the proximity sensor 316.

The controller sub-system 130 may in turn transmit such obtained information to the computing device 100B via the wireless interface 160 (e.g., Bluetooth or WiFi connection) or the wired interface 170 (e.g., USB connection).

In one embodiment, the stimulus control application of the computing device 100B obtains some or all of the environmental contextual data based on built-in sensors on the computing device 100B or based on data already accessible on the computing device 100B. For instance, the stimulus control application of the computing device 100B may:
obtain the time of day and the current geographical location by accessing system data (e.g., clock and/or location data) available on the computing device 100B,
obtain the ambient natural light level, ambient artificial light level, and/or ultraviolet light level by accessing output data from a light sensor and/or camera on the computing device 100B,
obtain the ambient noise level and/or proximity to other individuals by accessing audio data from a microphone on the computing device 100B, and
obtain the local outdoor temperature, absolute/relative humidity, and/or weather information at the current geographical location by (i) accessing location data available on the computing device 100B (e.g., via a built-in GPS sensor) and (ii) accessing an Internet portal that provides location-based temperature and/or weather information.

In one embodiment, the stimulus control application of the computing device 100B obtains some or all of the environmental contextual data from the peripheral sensor set 100C. For instance, various sensors within the peripheral sensor set 100C may be in wireless (e.g., WiFi or Bluetooth) or wired (e.g., USB) communication with the computing device 100B and may transmit sensor data to the computing device 100B.

In one embodiment, the stimulus control application of the computing device 100B obtains some or all of the environmental contextual data from the Internet. For instance, the stimulus control application may:
obtain the time of day and the current geographical location by accessing such information on the Internet (e.g., an Internet portal providing time information and general geographical location based on IP address), and
obtain the local outdoor temperature, absolute/relative humidity, and/or weather information at the current geographical location by accessing an Internet portal that provides location-based temperature and/or weather information.

In addition (or as an alternative), other applications executed on the computing device 100B may collect environmental contextual data, and provide such data to a server (e.g., via the Internet). The stimulus control application may then download such collected contextual data from the Internet.

In one embodiment, the stimulus control application of the computing device 100B obtains some or all of the environmental contextual data from third-party app data input, either maintained internally within the computing device 100B or from the Internet.

In step S620, the stimulus control application of the computing device 100B obtains personal contextual data, such as biometric personal contextual data and/or non-biometric personal contextual data. In one embodiment, the obtained biometric personal contextual data includes one or more of EMG measurements, EEG measurements, heart rate, heart rate variability, oxygen saturation level, galvanic skin response, blood pressure, body temperature, glucose level, respiratory rate, hormone levels, and sleep data. The obtained biometric personal contextual data may also include fitness data (e.g., daily step count). The obtained non-biometric personal contextual data may include a user's personal calendar events.

In one embodiment, the stimulus control application of the computing device 100B obtains some or all of the biometric personal contextual data from outputs from sensors within the biometric sensor set 110-2 of the stimulus delivery device 100A. For instance, the controller sub-system 130 may:
obtain a user's EMG measurement from the EMG sensor 320,
obtain a user's EEG measurement from the EEG sensor 321,
obtain a user's heart rate and/or heart rate variability from the heart rate/variability sensor 322,
obtain a user's oxygen saturation from the oxygen saturation sensor 323,
obtain a user's galvanic skin response from the galvanic skin response sensor 324,
obtain a user's blood pressure level from the blood pressure sensor 325,
obtain a user's body temperature from the body temperature sensor 326, and
obtain a user's blood glucose level from the blood glucose sensor 327.

The controller sub-system 130 may in turn transmit such obtained information to the computing device 100B via the wireless interface 160 (e.g., Bluetooth or WiFi connection) or the wired interface 170 (e.g., USB connection).

In one embodiment, the controller sub-system 130 of the stimulus delivery device 100A obtains some or all of the personal contextual data based on built-in sensors on the computing device 100B or based on data already accessible on the computing device 100B. For instance, the stimulus control application executed on the computing device 100B may:
obtain a user's heart rate, heart rate variability, oxygen saturation, and blood glucose level based on outputs from built-in sensors on the computing device 100B, and/or
obtain a user's calendar events from a calendar application on the computing device 100B.

In one embodiment, the stimulus control application of the computing device 100B obtains some or all of the biometric personal contextual data from the peripheral sensor set 100C. For instance, various sensors within the peripheral sensor set 100C may be in wireless (e.g., WiFi or Bluetooth) or wired (e.g., USB) communication with the computing device 100B and may transmit sensor data to the computing device 100B. Various examples of such sensors that may be encompassed within the peripheral sensor set 100C include, but are not limited to, smartwatches, fitness bands/trackers, sleep trackers, and heart rate monitors that may communicate with the computing device 100B via WiFi, Bluetooth and/or ANT+protocols.

In one embodiment, the stimulus control application of the computing device 100B obtains some or all of the personal contextual data from the Internet. For instance, where a user utilizes a sensor within the peripheral sensor set 100C (e.g., fitness tracker) where collected biometric data is provided to an Internet server and is accessible via an Internet portal (e.g., by Apple, Fitbit, Garmin, etc.), the stimulus control application of the computing device 100B may obtain some or all of the biometric personal contextual data by accessing the Internet portal. And, where a user has a personal calendar accessible via an Internet portal, the stimulus control application of the computing device 100B may obtain some or all of the calendar data by accessing the Internet portal.

In one embodiment, the stimulus control application of the computing device 100B obtains some or all of the personal contextual data from third-party app data input, either maintained internally within the computing device 100B or from the Internet.

It will be appreciated that the obtained biometric personal contextual data may include current (e.g., real-time) biometric data, historical biometric data, or a combination of both.

In step S630, the stimulus control application of the computing device 100B selects an ideal content program, based on the obtained environmental and personal contextual data. It will be appreciated that such selection may be implemented based on a variety of approaches. For example, based on the obtained local time, a content program having a particularly higher or lower dosage of blue light may be selected, given that blue light may interfere with natural circadian rhythms and cause insomnia. Based on the obtained ambient natural, ambient artificial, or ultraviolet light levels, a content program having a particularly higher or lower dosage intensity of light may be selected. Based on the obtained ambient noise level or proximity information, a content program having a particularly higher or lower audio intensity or range may be selected. Based on EMG, EEG, heart rate, blood pressure, or respiratory rate, a content program designed to provide a calming or soothing experience or, conversely, a content program designed to increase alertness and/or reduce drowsiness, may be selected.

In one embodiment, the stimulus control application of the computing device 100B determines an ideal content program by transmitting collected contextual information to a server (e.g., via the Internet) and receiving an identification of a content program already stored on the controller sub-system 130 or the computing device 100B and/or receives the content program itself.

In step S640, the stimulus control application of the computing device 100B controls the controller sub-system 130 which, in turn, controls the emitter sub-system 120 to emit light and/or sound according to the selected content program.

### Methods of Adapting Existing Content using Contextual Input(s)

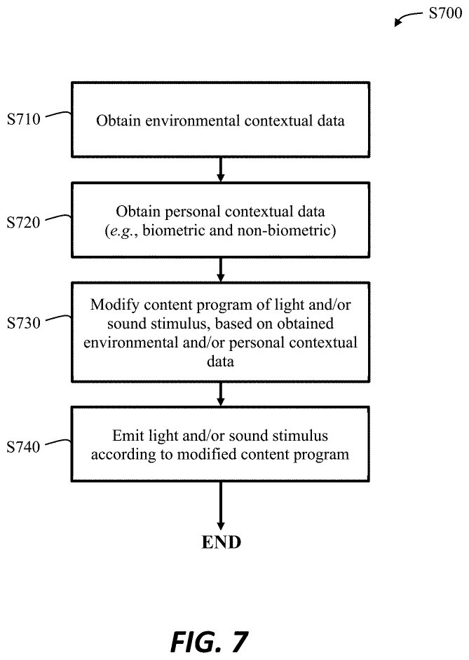

Various methods of adapting existing content based on contextual input(s) according to the invention will now be described. FIG. 7 illustrates an embodiment of a process S700 for adapting existing content based on contextual input(s), in accordance with the invention.

First, in step S710, the controller sub-system 130 of the stimulus delivery device 100A obtains environmental contextual data, in the same manner as step S510 described above,

In step S720, the controller sub-system 130 obtains personal contextual data, in the same manner as step S520 described above,

In step S730, the controller sub-system 130 modifies an existing content program based on the obtained environmental and personal contextual data. It will be appreciated that such modification may be implemented based on a variety of approaches. For example, based on the obtained local time, a content program may be modified to increase or decrease a dosage of blue light, given that blue light may interfere with natural circadian rhythms and cause insomnia. Based on the obtained ambient natural, ambient artificial, or ultraviolet light levels, a content program may be modified to increase or decrease a dosage intensity of light. Based on the obtained ambient noise level or proximity information, a content program may be modified to increase or decrease an audio intensity or range. Based on EMG, EEG, heart rate, blood pressure, or respiratory rate, a content program may be modified to provide a more calming or soothing experience or, conversely, may be modified to increase alertness and/or reduce drowsiness. It will be appreciated that any of the parameters discussed above with respect to the light emitter sub-system 140 and/or the sound emitter sub-system 150 may be modified based on any of the contextual data.

In step S740, the controller sub-system 130 controls the emitter sub-system 120 to emit light and/or sound according to the modified content program.

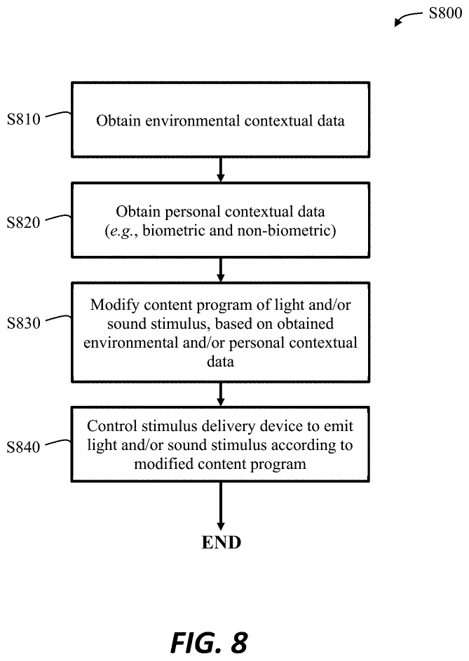

FIG. 8 illustrates an embodiment of another process S800 for adapting existing content based on contextual input(s), in accordance with the invention. The process S800 differs from the process S700 primarily in that the computing device 100B, instead of the controller sub-system 130, performs many of the steps. For instance, a stimulus control application running on the computing device 100B performs many of the steps.

First, in step S810, the stimulus control application of the computing device 100B obtains environmental contextual data, in the same manner as step S610 described above,

In step S820, the stimulus control application of the computing device 100B obtains personal contextual data, in the same manner as step S620 described above,

In step S830, the stimulus control application of the computing device 100B modifies an existing content program based on the obtained environmental and personal contextual data. It will be appreciated that such modification may be implemented based on a variety of approaches. For example, based on the obtained local time, a content program may be modified to increase or decrease a dosage of blue light, given that blue light may interfere with natural circadian rhythms and cause insomnia. Based on the obtained ambient natural, ambient artificial, or ultraviolet light levels, a content program may be modified to increase or decrease a dosage intensity of light. Based on the obtained ambient noise level or proximity information, a content program may be modified to increase or decrease an audio intensity or range. Based on EMG, EEG, heart rate, blood pressure, or respiratory rate, a content program may be modified to provide a more calming or soothing experience or, conversely, may be modified to increase alertness and/or reduce drowsiness. It will be appreciated that any of the parameters discussed above with respect to the light emitter sub-system 140 and/or the sound emitter sub-system 150 may be modified based on any of the contextual data.

In step S840, the stimulus control application of the computing device 100B controls the controller sub-system 130 which, in turn, controls the emitter sub-system 120 to and emit light and/or sound according to the modified content program.

### Methods of Generating Content using Contextual Input(s)

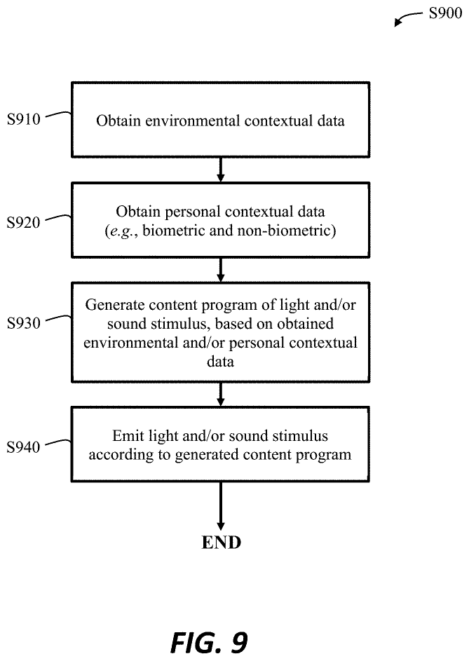

Various methods of generating content using contextual input(s) according to the invention will now be described. FIG. 9 illustrates a first embodiment of a process S900 for generating context based on contextual input(s), in accordance with the invention.

First, in step S910, the controller sub-system 130 of the stimulus delivery device 100A obtains environmental contextual data, in the same manner as step S510 described above,

In step S920, the controller sub-system 130 obtains personal contextual data, in the same manner as step S520 described above,

In step S930, the controller sub-system 130 generates a content program based on the obtained environmental and personal contextual data. It will be appreciated that such generation may be implemented based on a variety of approaches. For example, based on the obtained local time, a content program may be generated with a particularly higher or lower dosage of blue light, given that blue light may interfere with natural circadian rhythms and cause insomnia. Based on the obtained ambient natural, ambient artificial, or ultraviolet light levels, a content program may be generated with a particularly higher or lower dosage intensity of light. Based on the obtained ambient noise level or proximity information, a content program may be generated with a particularly higher or lower audio intensity or range. Based on EMG, EEG, heart rate, blood pressure, or respiratory rate, a content program may be generated having aspects believed to provide a more calming or soothing experience or, conversely, may be generated having aspects believed to increase alertness and/or reduce drowsiness. It will be appreciated that the generation of the content program generation may include control of some or all of the parameters discussed above with respect to the light emitter sub-system 140 and/or the sound emitter sub-system 150, based on any of the contextual data.

In step S940, the controller sub-system 130 controls the emitter sub-system 120 to emit light and/or sound according to the generated content program.

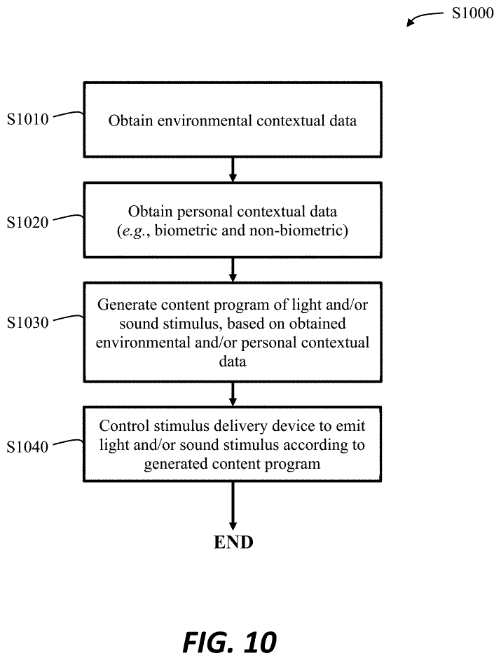

FIG. 10 illustrates an embodiment of another process S1000 for adapting existing content based on contextual input(s), in accordance with the invention. The process S1000 differs from the process S900 primarily in that the computing device 100B, instead of the controller sub-system 130, performs many of the steps. For instance, a stimulus control application running on the computing device 100B performs many of the steps.

First, in step S1010, the stimulus control application of the computing device 100B obtains environmental contextual data, in the same manner as step S610 described above,

In step S1020, the stimulus control application of the computing device 100B obtains personal contextual data, in the same manner as step S620 described above,

In step S1030, the stimulus control application of the computing device 100B generates a content program based on the obtained environmental and personal contextual data. It will be appreciated that such generation may be implemented based on a variety of approaches. For example, based on the obtained local time, a content program may be generated with a particularly higher or lower dosage of blue light, given that blue light may interfere with natural circadian rhythms and cause insomnia. Based on the obtained ambient natural, ambient artificial, or ultraviolet light levels, a content program may be generated with a particularly higher or lower dosage intensity of light. Based on the obtained ambient noise level or proximity information, a content program may be generated with a particularly higher or lower audio intensity or range. Based on EMG, EEG, heart rate, blood pressure, or respiratory rate, a content program may be generated having aspects believed to provide a more calming or soothing experience or, conversely, may be generated having aspects believed to increase alertness and/or reduce drowsiness. It will be appreciated that the generation of the content program generation may include control of some or all of the parameters discussed above with respect to the light emitter sub-system 140 and/or the sound emitter sub-system 150, based on any of the contextual data.

In step S1040, the stimulus control application of the computing device 100B controls the controller sub-system 130 which, in turn, controls the emitter sub-system 120 to and emit light and/or sound according to the generated content program.

### Non-limiting Examples of Content being Selected, Modified, and/or Generated Based on Contextual Data

Various non-limiting examples of the selection, modification, and/or generation of content based on contextual data will now be described. It will be appreciated that a variety of other examples beyond those discussed herein may be contemplated and are encompassed within the scope of the invention.

Example 1—In response to an obtained time of day indicating a late time, the system may (i) select content with a comparatively low level (or range) of blue light dosage and/or a comparatively high level (or range) of red and/or violet light dosage, (ii) modify existing content to decrease the level of blue light dosage and/or increase the level of red and/or violet light dosage, to present to a user, and/or (iii) generate content with a particular level (or range) of blue, red, and/or violet light dosage.

Example 2—In response to an obtained ambient natural light level indicating a low level, the system may (i) select content with a particular level (or range) of blue light dosage, (ii) modify the level of blue light dosage in existing content to present to a user, and/or (iii) generate content with a particular level (or range) of blue light dosage.

Example 3—In response to obtained recent sleep data indicating an off-target circadian rhythm, the system may (i) select content with a particular level (or range) of violet and/or blue light dosage, (ii) modify the level of violet and/or blue light dosage in existing content to present to a user, and/or (iii) generate content with a particular level (or range) of violet and/or blue light dosage. Using this approach, collected sleep data (e.g., from a fitness tracker) can be employed to control an appropriate dosage of violet light or blue light to be added to, or subtracted from, an experience with an objective of circadian alignment.

Example 4—In response to an obtained ambient natural light level indicating a high level, the system may (i) select content with a particular level (or range) of red light dosage, (ii) modify the level of red light dosage in existing content to present to a user, and/or (iii) generate content with a particular level (or range) of red light dosage. Using this approach, the ambient natural light level can be employed to control an appropriate dosage of red light (e.g., addition of combinations of different red wavelengths) to present a subconscious sensation of shade.

Example 5—In response to an obtained ambient artificial light level indicating a high level, the system may (i) select content with a particular level (or range) of violet and/or green light dosage, (ii) modify the level of violet and/or green light dosage in existing content to present to a user, and/or (iii) generate content with a particular level (or range) of violet and/or green light dosage. Using this approach, the ambient artificial light level can be employed to control an appropriate dosage of violet and/or green light (e.g., certain green and/or violet wavelengths), unavailable in most artificial light sources, to provide a subconscious sensation of a natural, outdoor environment.

Example 6—In response to obtained recent accelerometer, fitness activity, and/or heart rate level indicating recent physical activity/movement, the system may (i) select content with an increased level (or range) of theta and/or alpha brain frequencies for pulses of light or sound, and/or violet and/or deep red light dosages, (ii) modify the level of theta and/or alpha brain frequency dosages of light and/or sound, and/or violet and/or deep red light dosages, in existing content to present to a user, and/or (iii) generate content with a particular level (or range) of theta and/or alpha brain frequency dosages of light and/or sound, and/or violet and/or deep red light dosages. Using this approach, the accelerometer, fitness activity, and/or heart rate data can be employed to control appropriate dosages of theta and/or alpha brain frequencies, and/or violet and/or deep red light dosages, to provide, for example, a post-exercise cool-down experience, cueing gradual introduction to theta and alpha frequency stimuli in combination with increasing violet and deep red light to calm a user at a measured pace.

Example 7—In response to obtained recent accelerometer, fitness activity, and/or heart rate level indicating extended low physical activity/movement, the system may (i) select content with an increased level (or range) of alpha and/or beta brain frequency dosages of light and/or sound, and/or blue light dosages, (ii) modify the level of alpha and/or beta frequency brain dosages and/or blue light dosages, in existing content to present to a user, and/or (iii) generate content with a particular level (or range) of alpha and/or beta brain frequency dosages and/or blue light dosages. Using this approach, the accelerometer, fitness activity, and/or heart rate data can be employed to control appropriate dosages of alpha and/or beta brain frequency stimuli, and/or blue light dosages, to provide, for example, an energizing experience.

It will be appreciated that the foregoing examples, along with others that may be envisioned within the context of the present invention, may be used individually or in combination. For example, the addition/subtraction of violet and/or blue light dosage levels/ranges in example 3, influenced by obtained sleep data, could further be influenced by the time of day (example 1) and/or ambient natural light levels (example 2).

### Non-limiting Real-World Examples

Various real-world examples of the usage of the system will now be described.

As a first example, on a winter mid-afternoon where obtained environmental and personal contextual data indicates low ambient natural light (e.g., dense cloud cover), cold outdoor temperature (e.g., below 45 degrees Fahrenheit), and extended low user movement (e.g., based on accelerometer, fitness tracker, and/or heart rate data), the system may select a particular content with stimuli designed to help a user stay motivated and get moving (e.g., a faster pace musical score with alpha and beta light frequencies focused on blue light for alertness). Or, the system may modify an existing content selected by the user, to emphasize these stimuli characteristics. Or, the system may generate new content that promotes these stimuli characteristics.

As a second example, on a long summer evening following a day of sun for extended periods, the system may select a particular content with stimuli designed to “slow down” a user (e.g., slow and melodic musical score and transitions from faster, brighter light pulsation to dimmer, slower light pulsation). Or, the system may modify an existing content selected by the user, to emphasize these stimuli characteristics. Or, the system may generate new content that promotes these stimuli characteristics.

As a third example, for an evening session, the system may select a particular content with relatively low levels of blue light. Or, the system may modify an existing content selected by the user, to reduce the levels of blue light and/or replace the blue light with frequencies aligned to the phase of the sun setting transitioning to twilight.

As a fourth example, for a morning session prior to a calendar event entitled “tennis,” the system may select a particular content with elevated blue light levels (to awaken the user) or high binaural beat audio. Or the system may modify an existing content selected by the user, to increase blue light level, increase infrared light levels (e.g., to temporarily boost visual acuity), and/or increase the binaural audio (e.g., from 8-14 Hz (alpha frequency) to 14-30 Hz (beta frequency)). Or, the system may generate content with these features.

As a fifth example, where the collected data indicates a user's elevated heart rate after a morning full of meetings (based on full calendar time slots), the system may select a relaxation session that fits within the user's period of availability prior to the next calendar event. Or, the system may modify an existing content to shorten it to fit within the user's availability. Or, the system may generate a relaxation content that fits within the user's availability.

As a sixth example, where the collected data indicates that a user has racing thoughts before bedtime (e.g., as self-reported and/or confirmed through sensor data), the system may select a session having 4-8 Hz (theta) and/or 8-14 Hz (alpha) pulse frequencies and relatively high amber, red, and/or infrared light frequencies. Or, the system may modify an existing content to emphasize these properties, and/or add increasing variation in the content to help reduce rumination. Or, the system may generate new content that focuses on these properties.

Use of language herein such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (just X, or just Y, or just Z) and multiple items (i.e., {X and Y}, {X and Z}, {Y and Z}, or {X, Y, and Z}). “At least one of” is not intended to convey a requirement that each possible item must be present.

Further aspects of the present disclosure are provided by the subject matter of the following clauses.

A method of emitting a light stimulus experience, comprising: receiving at least one of (i) environmental contextual data and (ii) personal contextual data including biometric contextual data; and controlling, based on the at least one of the environmental contextual data and the personal contextual data, one or more light emitters to administer a dosage of light to a user.

The method according to any preceding clause, wherein the environmental contextual data includes one or more of time of day, weather, current geographical location, ambient natural light level, ambient artificial light level, ultraviolet light level, ambient noise level, ambient temperature, local outdoor temperature at the current geographical location, absolute and/or relative humidity, and proximity to other individuals.

The method according to any preceding clause, wherein the biometric contextual data includes one or more of EMG measurements, EEG measurements, heart rate, heart rate variability, oxygen saturation level, galvanic skin response, blood pressure, body temperature, glucose level, respiratory rate, hormone levels, sleep data, and fitness data.

The method according to any preceding clause, wherein the controlling includes controlling a light wavelength for the dosage of light, based on the environmental contextual data or the personal contextual data.

The method according to any preceding clause, wherein the controlling includes controlling a light intensity for the dosage of light, based on the environmental contextual data or the personal contextual data.

The method according to any preceding clause, wherein the controlling includes controlling an emission duration for the dosage of light, based on the environmental contextual data or the personal contextual data.

The method according to any preceding clause, wherein the environmental contextual data includes at least one of a time of day and an ambient natural light level, and wherein the controlling includes controlling an amount of blue light for the dosage of light based on the at least one of the time of day and the ambient natural light level.

The method according to any preceding clause, wherein the personal contextual data includes at least one of sleep data and fitness data, and wherein the controlling includes controlling the dosage of light based on the at least one of the sleep data and the fitness data.

The method according to any preceding clause, wherein the controlling includes selecting, based on the at least one of the environmental contextual data and the personal contextual data, a control sequence out of a plurality of predetermined control sequences for controlling the one or more light emitters.

The method according to any preceding clause, further comprising controlling, based on the at least one of the environmental contextual data and the personal contextual data, an auditory emitter to administer auditory stimulus to the user.

An apparatus comprising memory; and at least one processor, wherein the memory stores a computer program which, when executed by the at least one processor, causes the processor to: control, based on environmental contextual data corresponding to the apparatus or personal contextual data corresponding to a user, one or more light emitters to administer a dosage of light to the user.

The apparatus according to any preceding clause, wherein the apparatus includes the one or more light emitters.

The apparatus according to any preceding clause, wherein the one or more light emitters are provided in a second apparatus separate from the apparatus, wherein the apparatus further comprises a wireless data interface, and wherein the apparatus communicates with the second apparatus via the wireless data interface to control the one or more light emitters.

The apparatus according to any preceding clause, wherein the apparatus further comprises a wireless data interface, wherein the personal contextual data includes biometric contextual data, and wherein the computer program causes the processor to receive at least one of the environmental contextual data and the biometric contextual data via the wireless data interface.

The apparatus according to any preceding clause, wherein the controlling of the one or more light emitters includes controlling one or more of a light wavelength, a light intensity, and an emission duration for the dosage of light, based on the environmental contextual data or the personal contextual data.

The apparatus according to any preceding clause, wherein the environmental contextual data includes one or more of time of day, weather, current geographical location, ambient natural light level, ambient artificial light level, ultraviolet light level, ambient noise level, ambient temperature, local outdoor temperature at the current geographical location, absolute and/or relative humidity, and proximity to other individuals.

The apparatus according to any preceding clause, wherein the personal contextual data includes one or more of EMG measurements, EEG measurements, heart rate, heart rate variability, oxygen saturation level, galvanic skin response, blood pressure, body temperature, glucose level, respiratory rate, hormone levels, sleep data, and fitness data.

The apparatus according to any preceding clause, wherein the environmental contextual data includes at least one of a time of day and an ambient natural light level, and wherein the controlling of the one or more light emitters includes controlling an amount of blue light for the dosage of light based on the at least one of the time of day and the ambient natural light level.

The apparatus according to any preceding clause, wherein the personal contextual data includes at least one of sleep data and fitness data, and wherein the controlling of the one or more light emitters includes controlling the dosage of light based on the at least one of the sleep data and the fitness data.

The apparatus according to any preceding clause, wherein the controlling of the one or more light emitters includes selecting, based on the at least one of the environmental contextual data and the personal contextual data, a control sequence out of a plurality of predetermined control sequences for controlling the one or more light emitters.

The apparatus according to any preceding clause, wherein the computer program causes the processor to control, based on the environmental contextual data corresponding to the apparatus or the personal contextual data corresponding to the user, an auditory emitter to administer auditory stimulus to the user.

A non-tangible computer-readable medium storing a computer program which, when executed on a processor, performs steps comprising: receiving at least one of (i) environmental contextual data and (ii) personal contextual data including biometric contextual data; and controlling, based on the at least one of the environmental contextual data and the personal contextual data, one or more light emitters to administer a dosage of light to a user.

Although the foregoing description is directed to the embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

We claim:

1. A method of emitting a light stimulus experience, comprising:
receiving at least one of (i) environmental contextual data and (ii) personal contextual data including biometric contextual data; and
controlling, based on the at least one of the environmental contextual data and the personal contextual data, one or more light emitters to administer a dosage of light to a user.

2. The method of claim 1, wherein the environmental contextual data includes one or more of time of day, weather, current geographical location, ambient natural light level, ambient artificial light level, ultraviolet light level, ambient noise level, ambient temperature, local outdoor temperature at the current geographical location, absolute and/or relative humidity, and proximity to other individuals.

3. The method of claim 1, wherein the biometric contextual data includes one or more of EMG measurements, EEG measurements, heart rate, heart rate variability, oxygen saturation level, galvanic skin response, blood pressure, body temperature, glucose level, respiratory rate, hormone levels, sleep data, and fitness data.

4. The method of claim 1, wherein the controlling includes controlling a light wavelength for the dosage of light, based on the environmental contextual data or the personal contextual data.

5. The method of claim 1, wherein the controlling includes controlling a light intensity for the dosage of light, based on the environmental contextual data or the personal contextual data.

6. The method of claim 1, wherein the controlling includes controlling an emission duration for the dosage of light, based on the environmental contextual data or the personal contextual data.

7. The method of claim 1, wherein the environmental contextual data includes at least one of a time of day and an ambient natural light level, and
wherein the controlling includes controlling an amount of blue light for the dosage of light based on the at least one of the time of day and the ambient natural light level.

8. The method of claim 1, wherein the personal contextual data includes at least one of sleep data and fitness data, and
wherein the controlling includes controlling the dosage of light based on the at least one of the sleep data and the fitness data.

9. The method of claim 1, wherein the controlling includes selecting, based on the at least one of the environmental contextual data and the personal contextual data, a control sequence out of a plurality of predetermined control sequences for controlling the one or more light emitters.

10. The method of claim 1, further comprising controlling, based on the at least one of the environmental contextual data and the personal contextual data, an auditory emitter to administer auditory stimulus to the user.

11. An apparatus comprising:
memory; and
at least one processor,
wherein the memory stores a computer program which, when executed by the at least one processor, causes the processor to:
control, based on environmental contextual data corresponding to the apparatus or personal contextual data corresponding to a user, one or more light emitters to administer a dosage of light to the user.

12. The apparatus of claim 11, wherein the apparatus includes the one or more light emitters.

13. The apparatus of claim 11, wherein the one or more light emitters are provided in a second apparatus separate from the apparatus,
wherein the apparatus further comprises a wireless data interface, and
wherein the apparatus communicates with the second apparatus via the wireless data interface to control the one or more light emitters.

14. The apparatus of claim 11, wherein the apparatus further comprises a wireless data interface,
wherein the personal contextual data includes biometric contextual data, and
wherein the computer program causes the processor to receive at least one of the environmental contextual data and the biometric contextual data via the wireless data interface.

15. The apparatus of claim 11, wherein the controlling of the one or more light emitters includes controlling one or more of a light wavelength, a light intensity, and an emission duration for the dosage of light, based on the environmental contextual data or the personal contextual data.

16. The apparatus of claim 11, wherein the environmental contextual data includes one or more of time of day, weather, current geographical location, ambient natural light level, ambient artificial light level, ultraviolet light level, ambient noise level, ambient temperature, local outdoor temperature at the current geographical location, absolute and/or relative humidity, and proximity to other individuals.

17. The apparatus of claim 11, wherein the personal contextual data includes one or more of EMG measurements, EEG measurements, heart rate, heart rate variability, oxygen saturation level, galvanic skin response, blood pressure, body temperature, glucose level, respiratory rate, hormone levels, sleep data, and fitness data.

18. The apparatus of claim 11, wherein the environmental contextual data includes at least one of a time of day and an ambient natural light level, and
wherein the controlling of the one or more light emitters includes controlling an amount of blue light for the dosage of light based on the at least one of the time of day and the ambient natural light level.

19. The apparatus of claim 11, wherein the personal contextual data includes at least one of sleep data and fitness data, and
wherein the controlling of the one or more light emitters includes controlling the dosage of light based on the at least one of the sleep data and the fitness data.

20. The apparatus of claim 11, wherein the controlling of the one or more light emitters includes selecting, based on the at least one of the environmental contextual data and the personal contextual data, a control sequence out of a plurality of predetermined control sequences for controlling the one or more light emitters.

21. The apparatus of claim 11, wherein the computer program causes the processor to control, based on the environmental contextual data corresponding to the apparatus or the personal contextual data corresponding to the user, an auditory emitter to administer auditory stimulus to the user.

22. A non-tangible computer-readable medium storing a computer program which, when executed on a processor, performs steps comprising:
receiving at least one of (i) environmental contextual data and (ii) personal contextual data including biometric contextual data; and
controlling, based on the at least one of the environmental contextual data and the personal contextual data, one or more light emitters to administer a dosage of light to a user.
